(19) United States
(12) Patent Application Publication
(10) Pub. No.: US 2013/0066288 A1
(43) Pub. Date: Mar. 14, 2013

(54) Washable, Antimicrobial, Breathable, Multi-Layered, Absorbent Sheet And Articles

(71) Applicant: Elizabeth Lizhi Lin, Singapore (SG)

(72) Inventor: Elizabeth Lizhi Lin, Singapore (SG)

(21) Appl. No.: 13/641,699

(22) PCT Filed: Apr. 19, 2010
(86) PCT No.: PCT/SG2010/000154

§ 371 Date: Oct. 17, 2012

(51) Int. Cl. A61F 13/538

(52) U.S. Cl. 604/360

(57) ABSTRACT

A washable, antimicrobial, breathable, multi-layered absorbent sheet and article, has an antimicrobial property, inhibiting the growth of bacterial. It is also laminated with a monolithic membrane that allows moisture to migrate through the pores in the membrane whilst forming a barrier to the flow of excess liquid such as would otherwise take place in the absence of the membrane, thus preventing the formation of wet patches to the outer clothing of the wearer and also keeping the wearer feeling more comfortable due to the reduction of humidity and heat inside.

### BACKGROUND FOR INVENTION

In-absorbent articles such as breast pads, diapers, sanitary napkins, pantiliners and other absorbent pads, bacteria in body fluid such as sweat, breast milk, urine and menstrual blood may proliferate, which may cause skin sore or inflammation. With the onset of bacterial growth, unpleasant odors may also be produced. Many of these absorbent articles offer little or no breathable or moisture dissipating properties, making the wearer very uncomfortable due to the dampness and heat collected. Here, techniques for providing such an absorbent article with breathable, antibacterial function have been known.

In according to the aspects of this invention, a washable, antimicrobial, breathable, multi-layered absorbent sheet and articles, that has incorporated some materials or fabrics that are woven with a specialized silver-containing substrate yarn, such as the substrate known as X-Static® available from Noble Fiber Technologies or incorporated during the manufacturing process, Nano-silver SilverSol™ of Nano Silver Manufacturing Sdn Bhd. The nano-silver, when in contact with bacteria and fungus will adversely affect cellular metabolism and inhibit cell growth, an antimicrobial property, inhibiting the growth of bacteria and fungi.

It is also laminated with a monolithic polytetrafluoroethylene or expanded polytetrafluoroethylene (PTFE /ePTFE) membrane allows moisture to migrate through the pores in the membrane whilst forming a barrier to the flow of excess liquid such as would otherwise take place in the absence of the membrane, thus preventing the formation of wet patches to the outer clothing of the wearer and also keeping the wearer feeling more comfortable due to the reduction of humidity and heat inside.

An extra removable absorbent layer before the sponge or optional sewn, woven or bonded outer aesthetically appealing layer may be included after the dissipating layer to enhance the overall look of the product in a material or fabric that would not affect the above stated functions.

In accordance to the washable, antimicrobial, breathable, multi-layered absorbent sheet and articles, it can be made with accessories or fabrics likewise to be manufactured into washable diapers, sanitary pads, breast cups, pants, shorts, nursing bras, sports bra, shirts or into anything similar that is worn by the wearer for the function as understood.

### DESCRIPTION

In accordance with the first aspect of this invention there is provided a washable, antimicrobial, breathable, multi-layered absorbent sheet and articles that includes a liquid-permeable, and, as the front face sheet, a woven or non-woven fabric formed of a hydrophilic fiber such as a rayon fiber, a pulp fiber, and a synthetic fiber, a porous plastic sheet, or such sample as microfleece for diaper or lycra for breast cups or sanitary product, other suitable fabric materials that is for the same purpose that touches the skin of the wearer that ‘wicks’ the moisture from the wearer's skin to the inside of the absorbing layer to operatively serve as a transfer layer between a wearer's skin and the inside of the absorbent pad whilst itself remaining substantially dry may also be used.

This material may be coated with SilverSol™ nano-silver from Nano Silver Manufacturing Sdn Bhd. or woven with a percentage of X-static® silver fibers yarns available from Noble Biomaterials, Inc. or any other suitable methods in an amount that is sufficient for the antimicrobial and anti-odor property to be effective It also it is to be understood that other suitable fibers or solutions that exhibit antimicrobial properties may also be used. This will inhibit the growth of microbes or bacteria on the skin of the wearer.

In accordance with a first aspect of this invention there is provided a washable, antimicrobial, breathable, multi-layered absorbent sheet and articles that includes an absorbing layer for operatively maintaining, or assisting in the maintenance of, the basic shape of the diaper, breast cup, sanitary product or similar. It may be a sponge or foam layer in the form of a layer of sponge or foam material of a type widely used in the industry and that is typically a blend of polyester and polyurethane that has the SilverSol™ nano-silver solution incorporated, during the manufacturing process in an amount sufficient to exert an antimicrobial effect on said skin of wearer as well as in the material itself, serves as a structural layer that operatively maintains, or assists in maintaining, the basic shape of the diaper or breast cup as well as a moisture absorbing layer. This sponge or foam may also be heat formed to a suitable shape for its purpose.

It is the unique characteristic of this silver-containing material or sponge that irrespective of where it is positioned in the construction, once the microorganism comes in contact with the silver, it is killed, thereby eliminating or reducing odor. This configuration provides favorable interaction with the host environment, thereby enabling “active” ions to be available as an effective and efficient anti-microbial system with regards to the use of silver.

The exposed silver can result in reduction in the residual problems associated with bacterial colonization and odor. Bacteria release ammonia as a waste product and this offensive byproduct of bacteria is one of the principal causative agents in human-based odors. The silver-containing layer's rapid bactericidal performance reduces the opportunity for bacteria to produce ammonia. The response to stimuli (in this case bacteria and moisture) provides a zone of inhibition against bacteria provided by the silver ions. As silver is also a highly conductive metal both electrically and thermally, it provides great thermal, moisture transfer, and anti-static properties.

It is understood that additional removable absorbing layers may be included between the fabric touching the wearer's skin and the absorbent sponge to increase the absorption as needed. This removable layer may also be treated or coated with SilverSol™ nano-silver from Nano Silver Manufacturing Sdn. Bhd. or woven with a percentage of X-static® silver fibers yarns available from Noble Biomaterials, Inc. if needed to perform the antimicrobial function on the wearer's skin.

In accordance to the second aspect of the invention, there is laminated to the absorbing layer, a monolithic micro-porous polytetrafluoroethylene or expanded polytetrafluoroethylene (PTFE /ePTFE) membrane; being porous, was selected to allow moisture to migrate through the pores in the membrane whilst forming a barrier to the flow of excess liquid such as would otherwise take place in the absence of the membrane.

It operatively serves to retain moisture on the inside thereof and allow transfer of moisture through the membrane and evaporation thereof on the outside of the membrane, to provide a transfer mechanism for moisture that includes the steps of absorption of moisture on one side of the membrane where there is a higher humidity, diffusion through the membrane, and desorption of moisture on the other side of the membrane where there is a lower humidity.

The monolithic micro-porous breathable membrane contains 9 billion microscopic pores per square inch. These pores are 20,000 times smaller than a water droplet but 700 times larger than a water vapor molecule, which makes the membrane completely waterproof while allowing water vapors to escape from the inside. Such examples are sold under a brand name GORE-TEX® by W.L Gore and associates. It is also to be understood that other suitable membrane that exhibit the similar properties that is sufficient for our application may also be used.

Further features of these aspects of the washable, antimicrobial, breathable, multi-layered absorbent sheet and articles, provide for a moisture dissipating layer on the outside of the of the absorbing layer and membrane. Wherein the monolithic micro-porous polytetrafluoroethylene or expanded polytetrafluoroethylene (PTFE/ePTFE) membrane would be laminated in the center of both the inner sponge absorbing layer and outer moisture dissipating layer as stated.

It is this reduction of humidity and moisture level in the inner layers that help keep the wearer feeling comfortable. Such example is that no extra nursing pads would be required if this invention is incorporated and manufactured in a nursing bra as moisture dissipated will keep the wearer substantially dry. Diaper wearers will feel the reduction of heat and humidity level inside the diapers.

It may also be incorporated with the conventional construction that may embody any suitable or conventional structures or reinforcing means, as may be required, such as, buttons, buckles, clips, zips and the like to manufacture the product that would be used by the wearer for its function.

It will be understood that the differential in humidity on the inside and outside of the membrane that is required to promote diffusion is created not only by evaporation on the outside but also by body heat on the inside.

In order that the invention may be more fully understood different embodiments thereof will now be described with reference to the accompanying drawings.

### SUMMARY OF DRAWINGS

FIG. 1. Is the front view of the washable, antimicrobial, breathable, multi-layered embodiment of the breast cup.

FIG. 2. Is the side view of the washable, antimicrobial, breathable, multi-layered embodiment of the breast cup.

FIG. 3. Is the cross-sectional view showing an embodiment of the washable, antimicrobial, breathable, multi-layered absorbent sheet.

FIG. 3.(1) Is a liquid-permeable, woven or non-woven fabric formed of a hydrophilic fiber that touches the skin of the wearer. It may be coated with SilverSol™ nano-silver or woven with a percentage of X-static® silver fibers yarns or any other suitable methods in an amount that is sufficient for the antimicrobial and anti-odor property to be effective on the skin of the wearer. It also it is to be understood that other suitable fibers or solutions that exhibit anti-microbial properties may also be used.

FIG. 3.(2) Is the optional removable or sewn absorbent layer that may be added to enhance the total absorption capability if needed.

FIG. 3.(3) Is the structural moisture absorbent layer of the washable, antimicrobial, breathable, multi-layered absorbent sheet and article, constructed by sponge or other suitable material, which is to be treated with SilverSol™ nano-silver during the manufacturing process in suitable amounts that is sufficient for the antimicrobial and anti-odor property to be effective. It also it is to be understood that other suitable fibers or solutions that exhibit anti-bacterial, anti-odor may also be used.

FIG. 3.(4) Is a monolithic micro-porous polytetrafluoroethylene or expanded polytetrafluoroethylene (PTFE/ePTFE) membrane that allows the water vapors to be diffused from the inside to the outer layer fabric thus keeping the wearer reasonable dryer on the inside, and also acting as a barrier to prevent excess liquid to leak out to the outer clothing. The membrane is a monolithic micro-porous membrane (PTFE) that allows the transfer of water vapor without letting excess liquid from penetrating to the outer garments.

FIG. 3.(5) is the outer fabric that aids in the diffusion of the moisture and also retaining and protecting the membrane.

FIG. 3.(6) is an optional sewn outer aesthetically appealing layer.

FIG. 4. Is the front view of the washable, antimicrobial, breathable, multi-layered absorbent normal or nursing bra article.

FIG. 5. Is the front view of the washable, antimicrobial, breathable, multi-layered absorbent sports bra article.

FIG. 6. Is the front view of the washable, antimicrobial, breathable, multi-layered absorbent sheet used in a nursing tank top with the absorbent sheet at the frontal breast area article.

FIG. 7. Is the front view of the washable, antimicrobial, breathable, multi-layered absorbent diaper, sanitary product or similar article.

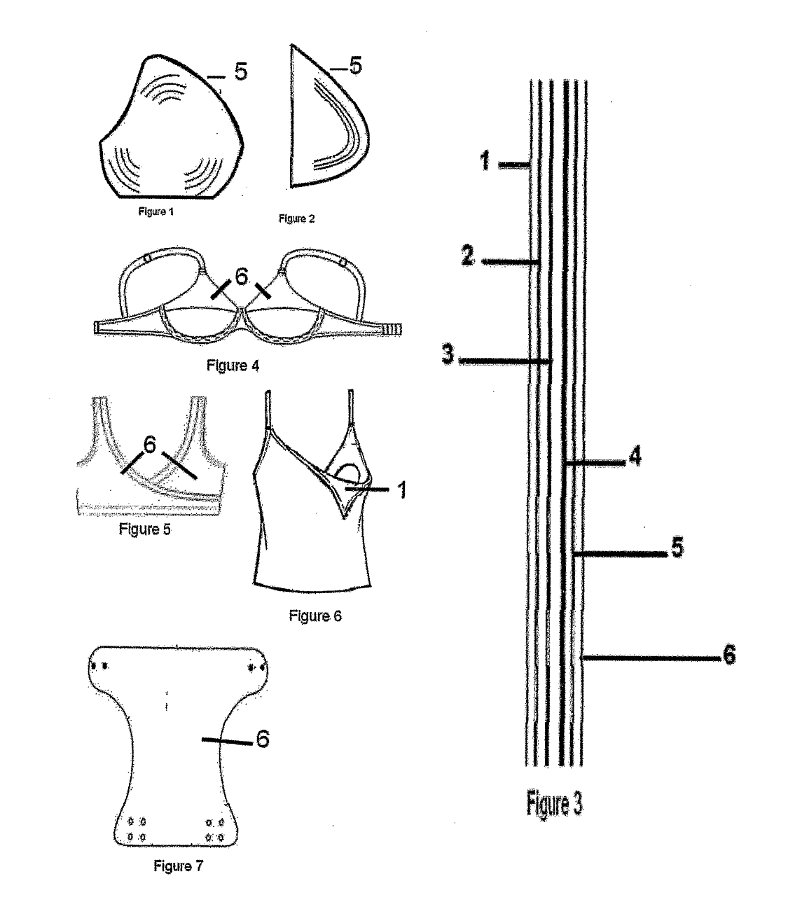

1. A washable, antimicrobial, breathable, multi-layered absorbent sheet and article that includes a woven or non-woven hydrophobic material that touches the skin of the wearer, material may be woven with a percentage of yarn coated with the X-static® silver fibers yarns available from Noble Biomaterials, Inc or coated with nano-silver under the trademark of SilverSol™ of Nano Silver Manufacturing Sdn. Bhd. in an amount sufficient to exert an antimicrobial effect on said skin of said wearer as well as in the fabric itself and that is used to wick the moisture from the body into the moisture absorbing sponge.

2. A washable, antimicrobial, breathable, multi-layered absorbent sheet and article that includes a structural layer for operatively maintaining, or assisting in the maintenance of, the basic shape diaper, breast cup, sanitary product or similar that acts also as an absorbing layer, this is a material of a type widely used in the industry and that is typically a blend of polyester and polyurethane that has incorporated the SilverSol™ nano-silver solution, during the manufacturing process in an amount sufficient to exert an antimicrobial effect on said skin of the wearer, serves as a structural layer that operatively maintains, or assists in maintaining, the basic shape of the diaper, breast cup or sanitary product or similar as well as acting as a moisture absorbing layer.

3. Additional removable or sewn absorbent materials may be added at layer (2) between the layer touching the skin and the sponge absorbing layer to increase the total absorption level of the diaper, breast cup or sanitary product or similar as needed.

4. A washable, antimicrobial, breathable, multi-layered absorbent sheet and article that in claims 1 and 2 and 3 may be woven, coated or treated with the X-static® or SilverSol™ in either one of the material or in all as long as it is sufficient for the antimicrobial property to be effective on the wearer's skin, it is also to be understood that other suitable fibers or solutions that exhibit anti-microbial properties may also be used.

5. A washable, antimicrobial, breathable, multi-layered absorbent sheet and article that has, laminated to the structural layer, a monolithic micro-porous polytetrafluoroethylene or expanded polytetrafluoroethylene (PTFE/ePTFE) membrane that operatively serves to retain moisture on the inside thereof and allow transfer of moisture through the membrane and evaporation thereof on the outside of the membrane, the diaper, breast cup, sanitary or similar product being characterised in that the membrane is an monolithic, scaffold membrane that is selected to provide a transfer mechanism for moisture that includes the steps of absorption of moisture on one side of the membrane where there is a higher humidity, diffusion through the membrane, and desorption of moisture on the other side of the membrane where there is lower humidity.

6. A washable, antimicrobial, breathable, multi-layered absorbent sheet and article that as claimed in claim 5, in which the membrane also serves as a protective layer where excess moisture present in the inner layer does not leak out to the outer layer of clothing.

7. A washable, antimicrobial, breathable, multi-layered absorbent sheet and article that has an outer layer that is laminated to the outside of the laminated sponge absorbent layer and membrane that serves as a moisture dissipating layer.

8. A washable, antimicrobial, breathable, multi-layered absorbent sheet and article as claimed in claim 7 in which the construction includes, an optional extra outer aesthetically appealing layer may be sewn on or used after the laminated dissipating layer.

9. A washable, antimicrobial, breathable, multi-layered absorbent sheet and article as claimed above, has antimicrobial properties that inhibits the growth of microbes, and has antibacterial properties to inhibit the growth of microbes on the wearer's skin.

10. A washable, antimicrobial, breathable, multi-layered absorbent sheet and article as claimed in any one of the preceding claims in which our invention may be manufactured in the form of a diaper, breast cup, sanitary product or similar accessory for removable installation in a supporting washable diaper, bra, or underwear or anything similar.

11. A washable, antimicrobial, breathable, multi-layered absorbent sheet and article as claimed that can be made with accessories or fabrics likewise to be manufactured into washable diapers, sanitary pads, breast cups, pants, shorts, nursing bras, sports bras, shirts or into anything similar that is worn by the wearer for the function as understood.

12. A washable, antimicrobial, breathable, multi-layered absorbent sheet and article that can be assembled into, or attached to, or used with any accessories that can or are used to make an item that is used for absorbing liquid whether or not the item manufactured is for the absorption of human waste or milk whether or not a torso encircling band or similar is used that can be assembled into, or attached to, or used in any other clothing or bras, embodying a pair of breast receiving cups, into a shirt, vest, sports bras or likewise anything similar that is used to cover the breasts or encircles the torso of a wearer.

13. A washable, antimicrobial, breathable, multi-layered absorbent sheet and article that may or may not be heat moulded to form the cup shape of a breast cup, diaper, sanitary pad or personal product as required.

14. A washable, antimicrobial, breathable, multi-layered absorbent sheet and article when manufactured into a breast cup in a nursing brassiere, can reduce the need for the lactating woman to use nursing pads as the dissipating property is sufficient to allow the wearer to feel substantially dry at the skin area of the breast touching the breast cup.

15. A washable, antimicrobial, breathable, multi-layered absorbent sheet and article when manufactured into a diaper or similar can allow the wearer to feel more comfortable due to the reduced humidity level and heat as compared to conventional washable diapers with plastic pants on.

16. It is also understood that other solutions, fabrics, materials or coatings, likewise or similar may be included or used into this invention to improve the function and esthetics or otherwise without effecting negatively the above stated claims.

17. It is also understood that the diaper shown and discussed in our illustration is not restricted to the above but also applies to pull up pants, incontinence pads or other such similar products not otherwise stated.

18. In particular, the antibacterial sheet is preferably used in an absorbent article for absorbing a body fluid such as washable diapers, breast cups, breast pads, bras, nursing tops with absorbent layers, sanitary napkin, pantiliners, vaginal or incontinence pads, each of which is used while being sewn or attached to the inner surface or made into the finished article as such.

19. The articles in this invention is understood to be the breast cup, sports bra, normal or nursing bra, diaper, sanitary napkins, pantiliners, incontinence pads or similar items that consist or manufactured with our inventive absorbent sheet.
